(19) United States
(12) Patent Application Publication
(10) Pub. No.: US 2016/0120407 A1
(43) Pub. Date: May 5, 2016

(54) MICROWAVE NEARFIELD RADAR IMAGING (NRI) USING DIGITAL BREAST TOMOSYNTHESIS (DBT) FOR NON-INVASIVE BREAST CANCER DETECTION

(71) Applicant: NORTHEASTERN UNIVERSITY, Boston, MA (US)

(72) Inventors: Jose Angel Martinez-Lorenzo, Boston, MA (US); Carey Rappaport, Wellesley, MA (US)

(21) Appl. No.: 14/897,621

(22) PCT Filed: Jun. 18, 2014
(86) PCT No.: PCT/US14/42842

§ 371 Date: Dec. 10, 2015

Related U.S. Application Data
(60) Provisional application No. 61/836,463, filed on Jun. 18, 2013.

(51) Int. Cl. A61B 5/00; A61B 6/02; A61B 6/00; A61B 5/05

(52) U.S. Cl. CPC A61B 5/0035; A61B 5/0507; A61B 6/025; A61B 6/502

(57) ABSTRACT

In some aspects, the disclosure is directed methods and systems for granular imaging of a distribution of tissues. A tomographic device may acquire a first image of a distribution of tissues, the first image including a plurality of pixels. A tomographic image processor may translate at least some of the plurality of pixels of the first image into a plurality of values representative of a distribution of dielectric constants corresponding to the distribution of tissues. A nearfield radar imaging (NRI) system may generate a second image of the distribution of tissues based on the plurality of values.

### RELATED APPLICATION

This application claims priority to U.S. Provisional Patent Application No. 61/836,463, entitled “Microwave Nearfield Radar Imaging (NRI) Using Digital Breast Tornosynthesis (DBT) for Non-Invasive Breast Cancer Detection”, filed Jun. 18, 2013, which is incorporated herein by reference in its entirety for all purposes.

### FIELD OF THE DISCLOSURE

This disclosure generally relates to systems and methods for performing radar-based imaging. In particular, this disclosure relates to systems and methods for performing radar-based imaging using data from a tomographic system.

### BACKGROUND OF THE DISCLOSURE

Conventional systems for imaging organic tissues, such as for detecting of breast cancer, may utilize X-ray technology. However, the range of radiological contrast provided by such systems, between normal tissue and diseased tissue, may be 1% or less. Radar-based imaging, on the other hand, may provide somewhat better radiological contrast. However, the random fibroglandular distribution in organic tissues generally overwhelms, masks or distorts the radiological features associated with diseased or cancerous tissue, making it challenging to distinguish the latter from other tissues.

### BRIEF SUMMARY OF THE DISCLOSURE

Described herein are systems and methods for performing microwave nearfield radar imaging (NRI) using digital breast tomosynthesis (DBT). Applications for the present systems and methods include non-invasive detection of anomalies such as breast cancer and certain diseased tissues. Embodiments of the present systems can incorporate an NRI system capable of providing significant radiological contrast (e.g., of the order of at least 10%) to distinguish fibroglandular tissue from cancer tissue. Large amounts of fibroglandular tissue, can introduce clutter which overwhelms or distorts the imaging of cancerous tissue or other anomalies. Rather than assuming a homogeneous distribution of fat tissue, the anomaly detection process of the NRI system may be improved or refined by the use of an accurate heterogeneous background comprising granular dielectric constant values. The NRI system can leverage on dielectric contrast between fat and other tissues, to perform modeling to facilitate clutter reduction. In certain embodiments, a complementary, integrated DBT system can scan the same tissue mass as the NRI and provide a high resolution, pixel-level distribution map of fat-content, which can be translated into a granular map of dielectric constant values for NRI processing, and result in improved cancerous and non-cancerous tissue differentiation. NRI imaging that has undergone such a process of clutter reduction can provide improved detection rates for anomalies.

In some aspects, the present disclosure pertains to a method for granular imaging of a distribution of tissues. The method may include acquiring, by a tomographic device, a first image of a distribution of tissues, the first image including a plurality of pixels. A tomographic image processor may translate at least some of the plurality of pixels of the first image into a plurality of values representative of a distribution of dielectric constants corresponding to the distribution of tissues. A nearfield radar imaging (NRI) system may generate a second image of the distribution of tissues based on the plurality of values.

In some embodiments, the distribution of tissues comprises breast tissues or some other portion of a human body. The NRI system may electromagnetically scan the distribution of tissues within a predetermined period of time from the acquisition of the first image. A support or compression system may maintain at least one of: a shape or a size of the distribution of tissues, across time periods during which the first image is acquired and during which the distribution of tissues is electromagnetically scanned by the NRI system. The support or compression system may locate the distribution of tissues within a container containing a bolus matching liquid.

In certain embodiments, the tomographic image processor may determine fat content corresponding to a first pixel of the plurality of pixels. The tomographic image processor may generate a first value of the plurality of values based on the determined fat content corresponding to the first pixel. The NRI system may generate a non-uniform function associated with at least one antenna of the NRI system. The NRI system may generate the non-uniform function based on a full wave electromagnetic method, i.e. finite difference frequency domain (FDFD) or finite element method (FEM). A user, algorithm or detector module may identify cancerous tissue or other feature from the second image.

In certain aspects, the present disclosure pertains to system for granular imaging of a distribution of tissues. The system may include a tomographic device. The tomographic device may be configured to acquire a first image of a distribution of tissues. The first image may include a plurality of pixels. The system may include a translation module. At least some of the plurality of pixels of the first image may be translated by the translation module into a plurality of values representative of a distribution of dielectric constants corresponding to the distribution of tissues. The system may include a nearfield radar imaging (NRI) system. The NRI system may be configured to generate a second image of the distribution of tissues based on the plurality of values.

In some embodiments, the tomographic device is configured to acquire the first image of the distribution of tissues, the distribution of tissues comprising breast tissues or some other portion of a human body. The NRI system may be configured to electromagnetically scan the distribution of tissues within a predetermined period of time from the acquisition of the first image. A compression or support structure may be configured to maintain at least one of: a shape or a size of the distribution of tissues, across time periods during which the first image is acquired and during which the distribution of tissues is electromagnetically scanned by the NRI system. The system may include a container within which the distribution of tissues is located, the container containing a bolus matching liquid.

A tomographic image processor may be configured to determine fat content corresponding to a first pixel of the plurality of pixels. The tomographic image processor may be configured to generate a first value of the plurality of values based on the determined fat content corresponding to the first pixel. The NRI system may be configured to generate a non-uniform function associated with at least one antenna of the NRI system. The NRI system may be configured to generate the non-uniform function based on a finite difference frequency domain (FDFD) method, finite element method (FEM) or any other full wave electromagnetic method. A user, algorithm or a detection system may use the second image to identify cancerous tissue or other feature.

The details of various embodiments of the invention are set forth in the accompanying drawings and the description below.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising client machines in communication with remote machines;

FIGS. 1B and 1C are block diagrams depicting embodiments of computing devices useful in connection with the methods and systems described herein;

FIG. 2A is a block diagram depicting one embodiment of a system for granular imaging of a distribution of tissues;

FIG. 2B is a block diagram depicting one embodiment of a tomographic device of a system for granular imaging of a distribution of tissues;

FIG. 2C is a block diagram depicting one embodiment of an NRI system of a system for granular imaging of a distribution of tissues;

FIG. 2D depicts example embodiments of representations of a distribution of tissues;

FIG. 2E depicts one embodiment of an electric field distribution superimposed on a composite model, and one embodiment of an optimized homogeneous geometry or model;

FIG. 2F depicts one embodiment of a difference between the electric field of the composite and the homogeneous models;

FIGS. 2G and 2H depict embodiments of plots of results obtained for average permittivity and dielectric constant;

FIGS. 2I and 2J depicts example embodiments of images including cancer tissue;

FIGS. 2K, 2L and 2M depict embodiments of configurations of an NRI system of a system for granular imaging of a distribution of tissues;

FIG. 2N depicts one embodiment of specifications for an embodiment of an NRI system of a system for granular imaging of a distribution of tissues; and

FIG. 2O shows one embodiment of a method for granular imaging of a distribution of tissues.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

For purposes of reading the description of the various embodiments below, the following descriptions of the sections of the specification and their respective contents may be helpful:
Section A describes a network environment and computing environment which may be useful for practicing embodiments described herein; and
Section B describes embodiments of systems and methods for microwave nearfield radar imaging (NRI) using digital breast tomosynthesis (DBT).

### A. Computing and Network Environment

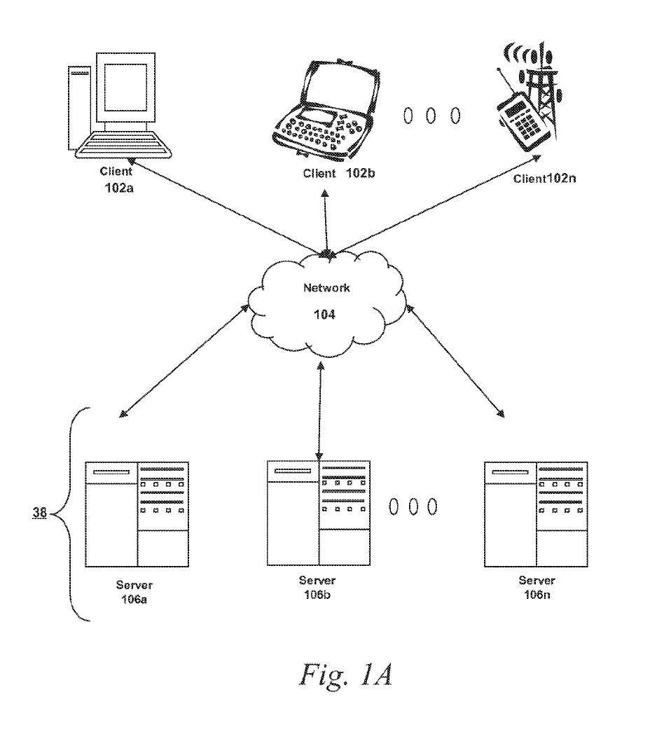

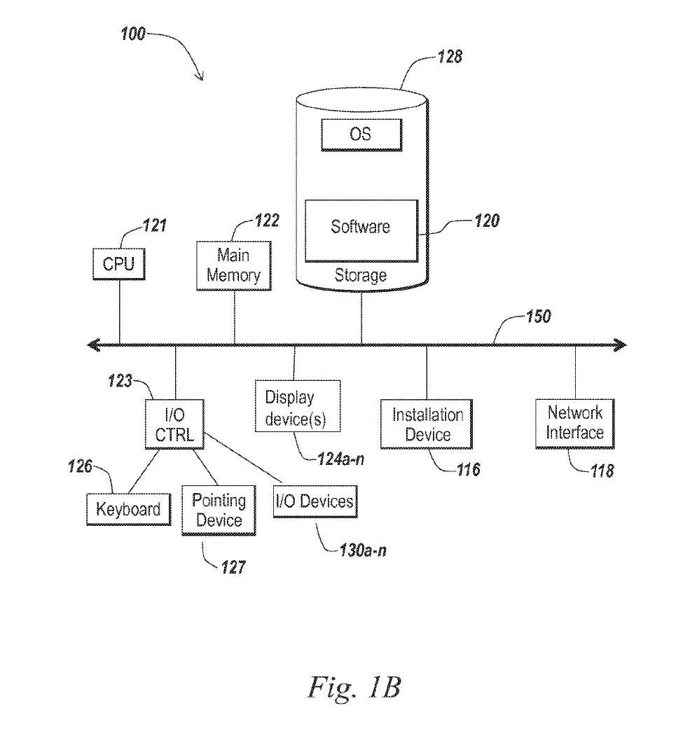

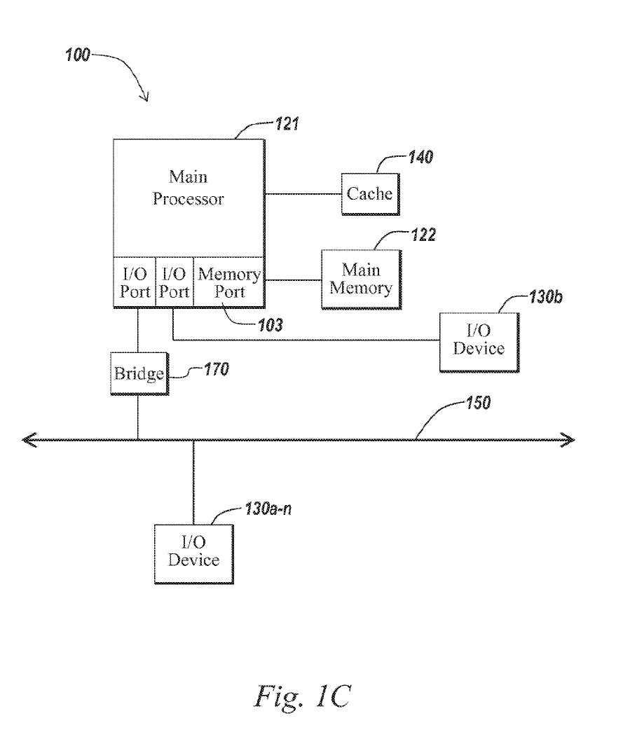

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 101a-101n (also generally referred to as local machine(s) 101, client(s) 101, client node(s) 101, client machine(s) 101, client computer(s) 101, client device(s) 101, endpoint(s) 101, or endpoint node(s) 101) in communication with one or more servers 106a-106n (also generally referred to as server(s) 106, node 106, or remote machine(s) 106) via one or more networks 104. In some embodiments, a client 101 has the capacity to function as both a client node seeking access to resources provided by a server and as a server providing access to hosted resources for other clients 101a-101n.

Although FIG. 1A shows a network 104 between the clients 101 and the servers 106, the clients 101 and the servers 106 may be on the same network 104. The network 104 can be a local-area network (LAN), such as a company Intranet, a metropolitan area network (MAN), or a wide area network (WAN), such as the Internet or the World Wide Web. In some embodiments, there are multiple networks 104 between the clients 101 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be any type and/or form of network and may include any of the following: a point-to-point network, a broadcast network, a wide area network, a local area network, a telecommunications network, a data communication network, a computer network, an ATM (Asynchronous Transfer Mode) network, a SONET (Synchronous Optical Network) network, a SDH (Synchronous Digital Hierarchy) network, a wireless network and a wireline network. In some embodiments, the network 104 may comprise a wireless link, such as an infrared channel or satellite band. The topology of the network 104 may be a bus, star, or ring network topology. The network 104 may be of any such network topology as known to those ordinarily skilled in the art capable of supporting the operations described herein. The network may comprise mobile telephone networks utilizing any protocol(s) or standard(s) used to communicate among mobile devices, including AMPS, TDMA, CDMA, GSM, GPRS, UMTS, WiMAX, 3G or 4G. In some embodiments, different types of data may be transmitted via different protocols. In other embodiments, the same types of data may be transmitted via different protocols.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS, manufactured by Microsoft Corp. of Redmond, Wash.), while one or more of the other servers 106 can operate on according to another type of operating system platform (e.g., Unix or Linux).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments. Hypervisors may include those manufactured by VMWare, Inc., of Palo Alto, Calif.; the Xen hypervisor, an open source product whose development is overseen by Citrix Systems, Inc.; the Virtual Server or virtual PC hypervisors provided by Microsoft or others.

In order to manage a machine farm 38, at least one aspect of the performance of servers 106 in the machine farm 38 should be monitored. Typically, the load placed on each server 106 or the status of sessions running on each server 106 is monitored. In some embodiments, a centralized service may provide management for machine farm 38. The centralized service may gather and store information about a plurality of servers 106, respond to requests for access to resources hosted by servers 106, and enable the establishment of connections between client machines 101 and servers 106.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

In one embodiment, the server 106 provides the functionality of a web server. In another embodiment, the server 106a receives requests from the client 101, forwards the requests to a second server 106b and responds to the request by the client 101 with a response to the request from the server 106b. In still another embodiment, the server 106 acquires an enumeration of applications available to the client 101 and address information associated with a server 106′ hosting an application identified by the enumeration of applications. In yet another embodiment, the server 106 presents the response to the request to the client 101 using a web interface. In one embodiment, the client 101 communicates directly with the server 106 to access the identified application. In another embodiment, the client 101 receives output data, such as display data, generated by an execution of the identified application on the server 106.

The client 101 and server 106 may be deployed as and/or executed on any type and form of computing device, such as a computer, network device or appliance capable of communicating on any type and form of network and performing the operations described herein. FIGS. 1B and 1C depict block diagrams of a computing device 100 useful for practicing an embodiment of the client 101 or a server 106. As shown in FIGS. 1B and 1C, each computing device 100 includes a central processing unit 121, and a main memory unit 122. As shown in FIG. 1B, a computing device 100 may include a storage device 128, an installation device 116, a network interface 118, an I/O controller 123, display devices 124a-101n, a keyboard 126 and a pointing device 127, such as a mouse. The storage device 128 may include, without limitation, an operating system and/or software. As shown in FIG. 1C, each computing device 100 may also include additional optional elements, such as a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, such as: those manufactured by Intel Corporation of Mountain View, Calif.; those manufactured by Motorola Corporation of Schaumburg, Ill.; those manufactured by International Business Machines of White Plains, N.Y.; or those manufactured by Advanced Micro Devices of Sunnyvale, Calif. The computing device 100 may be based on any of these processors, or any other processor capable of operating as described herein.

Main memory unit 122 may be one or more memory chips capable of storing data and allowing any storage location to be directly accessed by the microprocessor 121, such as Static random access memory (SRAM), Burst SRAM or SynchBurst SRAM (BSRAM), Dynamic random access memory (DRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Enhanced DRAM (EDRAM), synchronous DRAM (SDRAM), JEDEC SRAM, PC100 SDRAM, Double Data Rate SDRAM (DDR SDRAM), Enhanced SDRAM (ESDRAM), SyncLink DRAM (SLDRAM), Direct Rambus DRAM (DRDRAM), Ferroelectric RAM (FRAM), NAND Flash, NOR Flash and Solid State Drives (SSD). The main memory 122 may be based on any of the above described memory chips, or any other available memory chips capable of operating as described herein. In the embodiment shown in FIG. 1B, the processor 121 communicates with main memory 122 via a system bus 150 (described in more detail below). FIG. 1C depicts an embodiment of a computing device 100 in which the processor communicates directly with main memory 122 via a memory port 103. For example, in FIG. 1C the main memory 122 may be DRDRAM.

FIG. 1C depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1C, the processor 121 communicates with various I/O devices 130 via a local system bus 150. Various buses may be used to connect the central processing unit 121 to any of the I/O devices 130, including a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display 124, the processor 121 may use an Advanced Graphics Port (AGP) to communicate with the display 124. FIG. 1C depicts an embodiment of a computer 100 in which the main processor 121 may communicate directly with I/O device 130b, for example via HYPERTRANSPORT, RAPIDIO, or INFINIBAND communications technology. FIG. 1C also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices include keyboards, mice, trackpads, trackballs, microphones, dials, touch pads, and drawing tablets. Output devices include video displays, speakers, inkjet printers, laser printers, projectors and dye-sublimation printers. The I/O devices may be controlled by an I/O controller 123 as shown in FIG. 1B. The I/O controller may control one or more I/O devices such as a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices such as the USB Flash Drive line of devices manufactured by Twintech Industry, Inc. of Los Alamitos, Calif.

Referring again to FIG. 1B, the computing device 100 may support any suitable installation device 116, such as a disk drive, a CD-ROM drive, a CD-R/RW drive, a DVD-ROM drive, a flash memory drive, tape drives of various formats, USB device, hard-drive or any other device suitable for installing software and programs. The computing device 100 can further include a storage device, such as one or more hard disk drives or redundant arrays of independent disks, for storing an operating system and other related software, and for storing application software programs such as any program or software 120 for implementing (e.g., configured and/or designed for) the systems and methods described herein. Optionally, any of the installation devices 116 could also be used as the storage device. Additionally, the operating system and the software can be run from a bootable medium, for example, a bootable CD.

Furthermore, the computing device 100 may include a network interface 118 to interface to the network 104 through a variety of connections including, but not limited to, standard telephone lines, LAN or WAN links (e.g., 802.11, T1, T3, 56 kb, X.25, SNA, DECNET), broadband connections (e.g., ISDN, Frame Relay, ATM, Gigabit Ethernet, Ethernet-over-SONET), wireless connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, IEEE 802.11n, CDMA, GSM, WiMax and direct asynchronous connections). In one embodiment, the computing device 100 communicates with other computing devices 100′ via any type and/or form of gateway or tunneling protocol such as Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, card bus network adapter, wireless network adapter, USB network adapter, modem or any other device suitable for interfacing the computing device 100 to any type of network capable of communication and performing the operations described herein.

In some embodiments, the computing device 100 may comprise or be connected to multiple display devices 124a-124n, which each may be of the same or different type and/or form. As such, any of the I/O devices 130a-130n and/or the I/O controller 123 may comprise any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may comprise multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices, such as computing devices 100a and 100b connected to the computing device 100, for example, via a network. These embodiments may include any type of software designed and constructed to use another computer's display device as a second display device 124a for the computing device 100. One ordinarily skilled in the art will recognize and appreciate the various ways and embodiments that a computing device 100 may be configured to have multiple display devices 124a-124n.

In further embodiments, an I/O device 130 may be a bridge between the system bus 150 and an external communication bus, such as a USB bus, an Apple Desktop Bus, an RS-232 serial connection, a SCSI bus, a FireWire bus, a FireWire 800 bus, an Ethernet bus, an AppleTalk bus, a Gigabit Ethernet bus, an Asynchronous Transfer Mode bus, a FibreChannel bus, a Serial Attached small computer system interface bus, or a HDMI bus.

A computing device 100 of the sort depicted in FIGS. 1B and 1C typically operates under the control of operating systems, which control scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: Android, manufactured by Google Inc; WINDOWS 7 and 8, manufactured by Microsoft Corporation of Redmond, Wash.; MAC OS, manufactured by Apple Computer of Cupertino, Calif.; WebOS, manufactured by Research In Motion (RIM); OS/2, manufactured by International Business Machines of Armonk, N.Y.; and Linux, a freely-available operating system distributed by Caldera Corp. of Salt Lake City, Utah, or any type and/or form of a Unix operating system, among others.

The computer system 100 can be any workstation, telephone, desktop computer, laptop or notebook computer, server, handheld computer, mobile telephone or other portable telecommunications device, media playing device, a gaming system, mobile computing device, or any other type and/or form of computing, telecommunications or media device that is capable of communication. The computer system 100 has sufficient processor power and memory capacity to perform the operations described herein. For example, the computer system 100 may comprise a device of the IPAD or IPOD family of devices manufactured by Apple Computer of Cupertino, Calif., a device of the PLAYSTATION family of devices manufactured by the Sony Corporation of Tokyo, Japan, a device of the NINTENDO/Wii family of devices manufactured by Nintendo Co., Ltd., of Kyoto, Japan, or an XBOX device manufactured by the Microsoft Corporation of Redmond, Wash.

In some embodiments, the computing device 100 may have different processors, operating systems, and input devices consistent with the device. For example, in one embodiment, the computing device 100 is a smart phone, mobile device, tablet or personal digital assistant. In still other embodiments, the computing device 100 is an Android-based mobile device, an iPhone smart phone manufactured by Apple Computer of Cupertino, Calif., or a Blackberry handheld or smart phone, such as the devices manufactured by Research In Motion Limited. Moreover, the computing device 100 can be any workstation, desktop computer, laptop or notebook computer, server, handheld computer, mobile telephone, any other computer, or other form of computing or telecommunications device that is capable of communication and that has sufficient processor power and memory capacity to perform the operations described herein.

In some embodiments, the computing device 100 is a digital audio player. In one of these embodiments, the computing device 100 is a tablet such as the Apple IPAD, or a digital audio player such as the Apple IPOD lines of devices, manufactured by Apple Computer of Cupertino, Calif. In another of these embodiments, the digital audio player may function as both a portable media player and as a mass storage device. In other embodiments, the computing device 100 is a digital audio player such as an MP3 players. In yet other embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the communications device 101 includes a combination of devices, such as a mobile phone combined with a digital audio player or portable media player. In one of these embodiments, the communications device 101 is a smartphone, for example, an iPhone manufactured by Apple Computer, or a Blackberry device, manufactured by Research In Motion Limited. In yet another embodiment, the communications device 101 is a laptop or desktop computer equipped with a web browser and a microphone and speaker system, such as a telephony headset. In these embodiments, the communications devices 101 are web-enabled and can receive and initiate phone calls.

In some embodiments, the status of one or more machines 101, 106 in the network 104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Microwave Nearfield Radar Imaging Using Digital Breast Tomosynthesis

In certain embodiments, relying directly on DBT technology to detect breast cancer tissues embedded in fibroglandular tissue may be challenging, since the X-ray radiological contrast between these tissues is typically of the order of 1%. This can be challenging for intensity-based algorithms to detect breast cancers. Alternatively, by relying on image morphology, anomalies can occasionally be missed even by trained radiologists. In some embodiments, microwave NRI technology are used in a stand-alone mode, and may not be as useful for detecting cancer in heterogeneous breast volumes as the present methods and systems. Typical NRI imaging algorithms may assume a homogeneous background (e.g., a homogeneous low water content adipose background tissue distribution with an embedded isolated high water content tumor anomaly) inside of the breast as a first guess for image reconstruction. However, typical breast tissues, for example, can include significant amounts of finely distributed high water content fibroglandular tissue interspersed in the adipose tissue. As such, the simple inversion process of NRI processing may fail, and such a first guess is generally so unrealistic that the imaging algorithms may fail to find cancerous tissues.

Described herein are systems and methods for performing microwave nearfield radar imaging (NRI) using digital breast tomosynthesis (DBT). Applications for the present systems and methods include non-invasive detection of anomalies such as breast cancer and certain diseased tissues.

Embodiments of the present systems can incorporate an NRI system capable of providing significant radiological contrast (e.g., of the order of at least 10%) to distinguish fibroglandular tissue from cancer tissue. Large amounts of fibroglandular tissue, can introduce clutter which overwhelms or distorts the imaging of cancerous tissue or other anomalies. Rather than assuming a homogeneous distribution of fat tissue, the anomaly detection process of the NRI system may be improved or refined by the use of an accurate heterogeneous background comprising granular dielectric constant values. The NRI system can leverage on dielectric contrast between fat and other tissues, to perform modeling to facilitate clutter reduction. In certain embodiments, a complementary, integrated DBT system can scan the same tissue mass as the NRI and provide a high resolution, pixel-level distribution map of fat-content, which can be translated into a granular map of dielectric constant values for NRI processing, and result in improved cancerous and non-cancerous tissue differentiation. NRI imaging that have undergone such a process of clutter reduction can provide improved detection rates for anomalies.

This disclosure may include descriptions specific to detection of breast cancer, merely by way of illustration and not intended to be limiting in any way. However, it is noted that improving breast cancer screening accuracy is important for the public health service infrastructure. The present systems and methods can, for example contribute to the accurate identification of cancer without significantly increasing screening cost and time. For example, the present systems and methods may reduce false alarm rates and associated need for additional rounds of examination, and may allow for early treatment planning. It is noted that, according to the Centers for Disease Control and Prevention, breast cancer is the most common cancer among women, with a rate of 123.1 cases per 100,000, and it is the second leading cause of cancer death among women, with a rate of 22.2 cases per 100,000. In order to reduce the mortality rate, improvements over conventional mammography (CM) to detect breast cancer at earlier stages during regular screening procedures would be helpful. The sensitivity of mammography is typically 85-90%, meaning that 10-15% of cancers can go undetected and present themselves within one year after the mammogram. In the United States for example, approximately 45 million mammograms are performed annually with false positive rates of 8% to 10%. This means that approximately 4.5 million callback examinations at approximately $300/each are required to diagnose the cancer with certainty, making the total costs for callback exams $1.35 Billion annually. This figure can be magnified as emerging countries in Asia, the Middle East and Africa are added into the mix. In addition, there is also the unquantifiable human dimension with the unwanted stress and anxiety to patients receiving callbacks, of not knowing whether they have cancer. There is also the associated time delay in beginning treatment due to the delay of scheduling the second mammogram. For aggressive forms of breast cancer, even a one-month delay can have negative impact on patient outcomes.

Tomographic methods, such as digital breast tomosynthesis (DBT), can outperform CM by producing volumetric imaging, despite the limited radiological contrast of cancer to healthy tissue. Early diagnosis of breast cancer is believed to be an important factor in initiating treatment before the cancerous tissue becomes malignant or metastic. In accordance with some embodiments of the present methods and systems, a microwave NRI/DBT dual-mode imager can help in differentiating cancer tissue from the fibroglandular tissue with more certainty, making earlier detection more feasible.

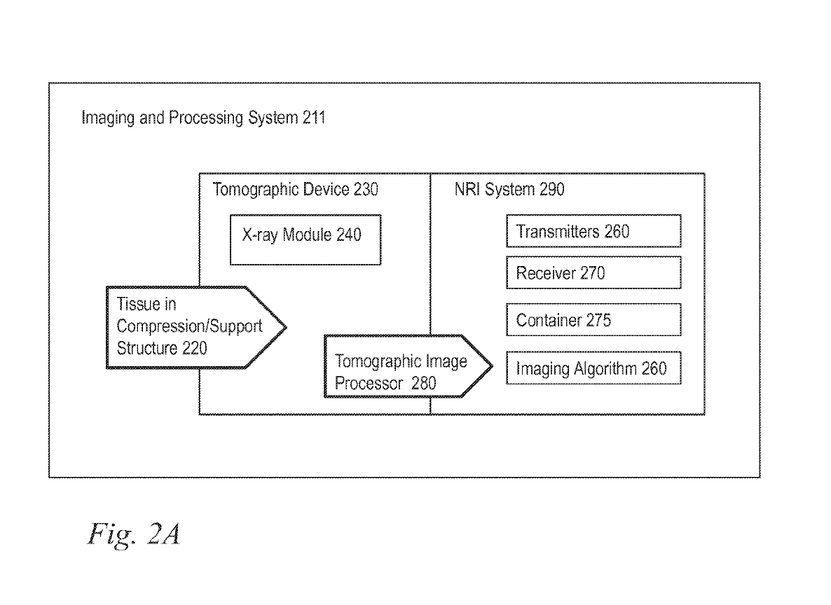

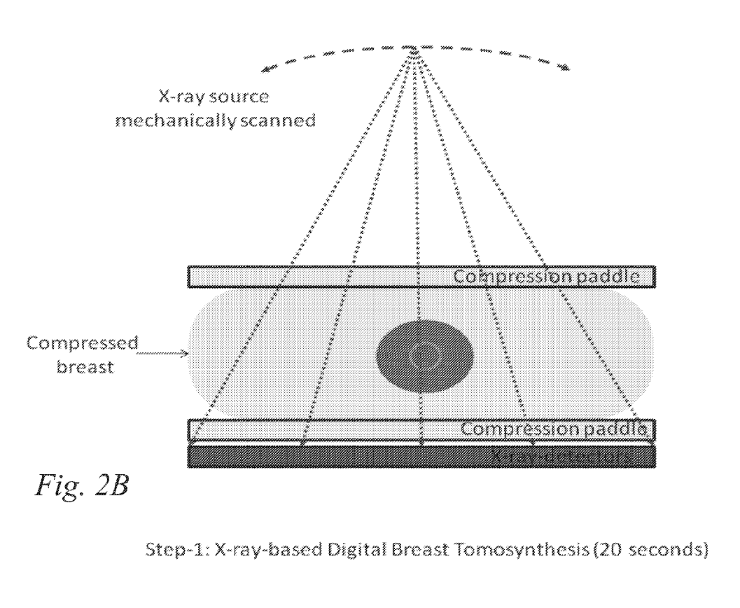

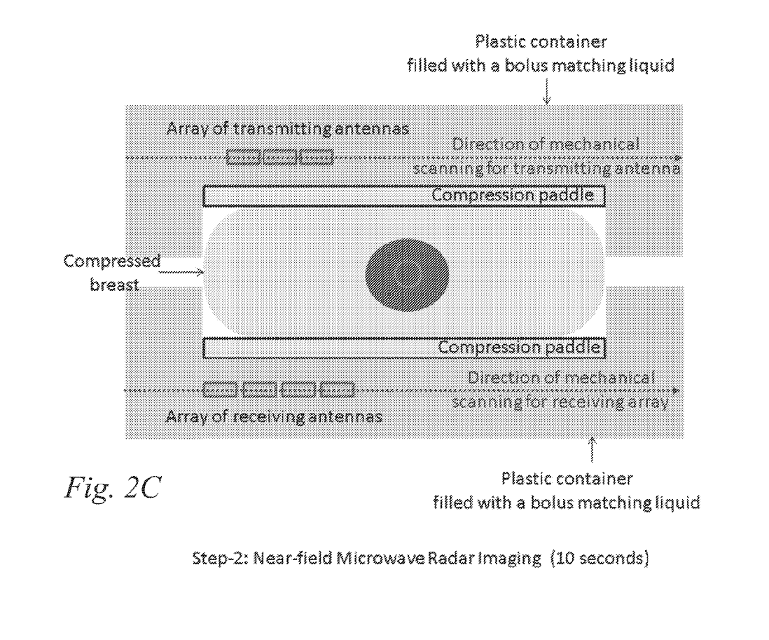

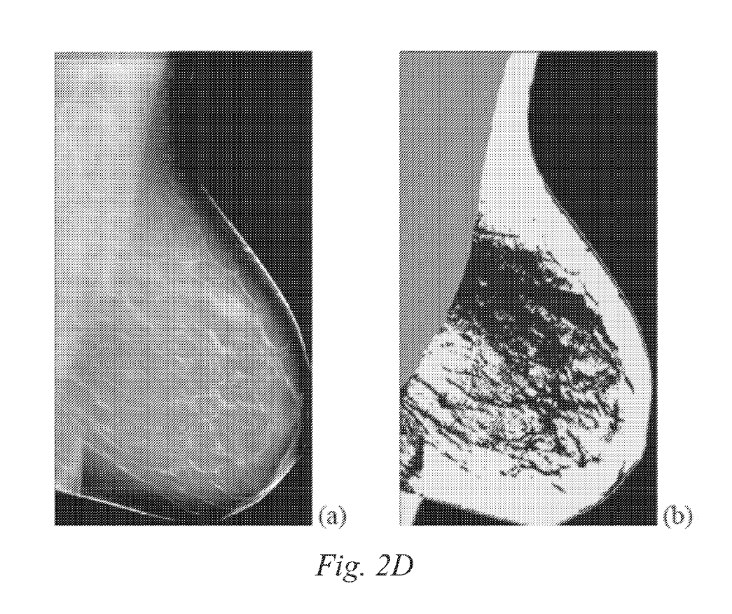

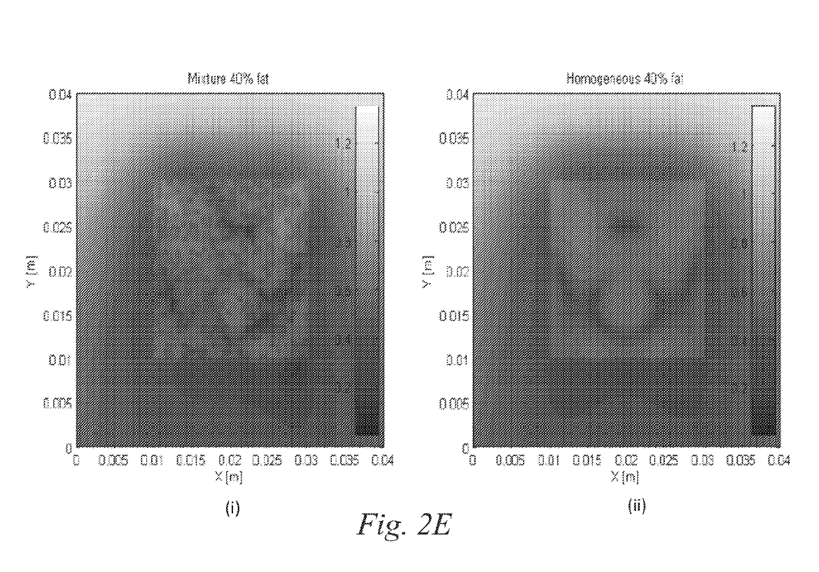

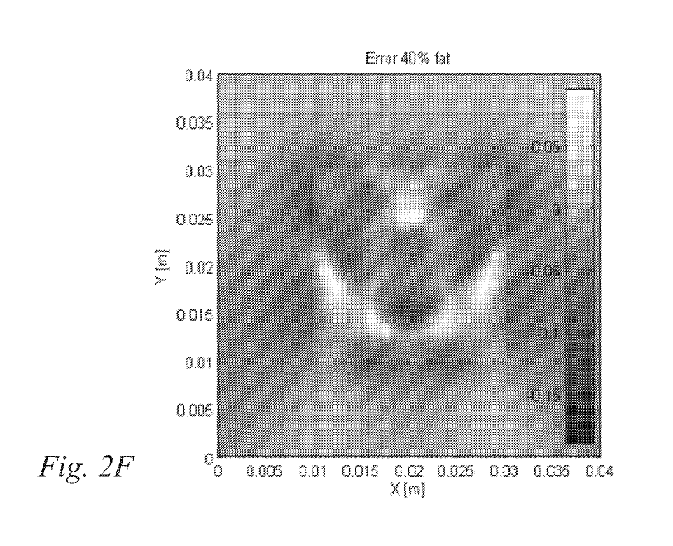

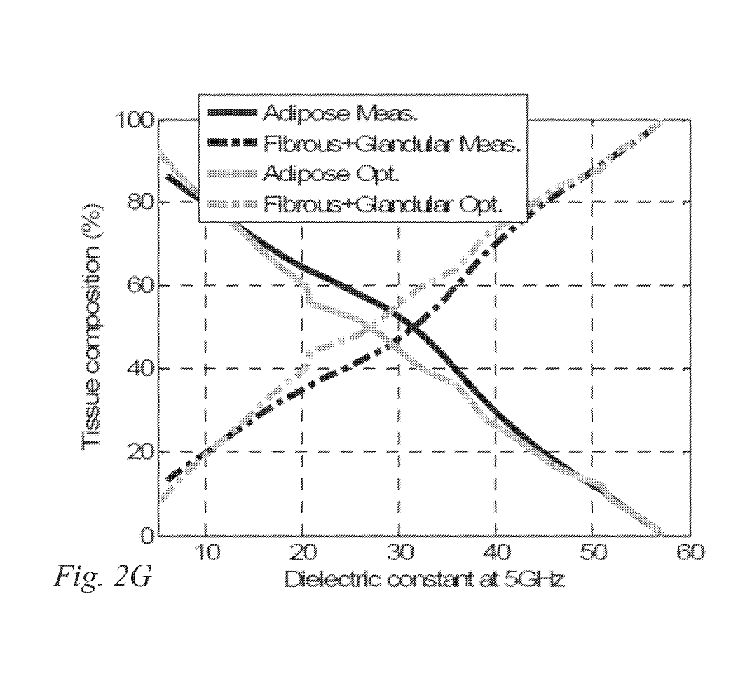

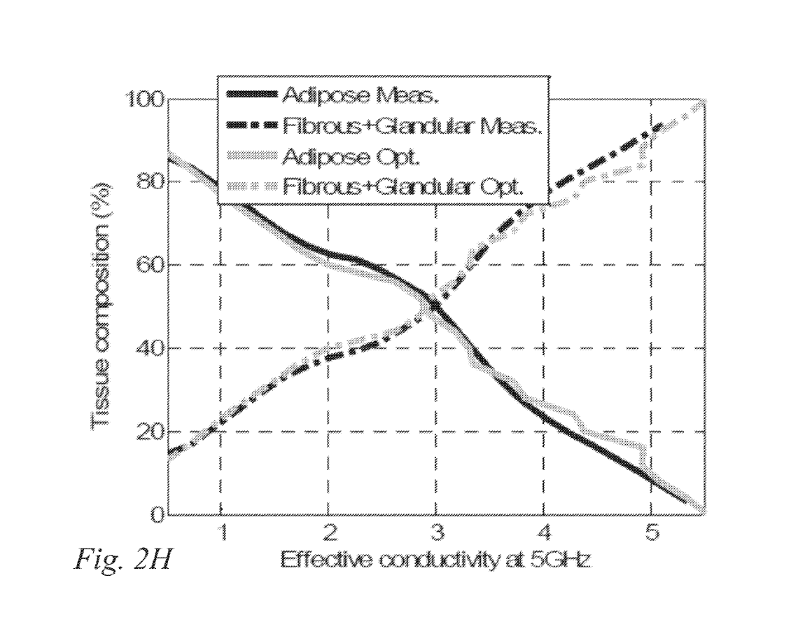

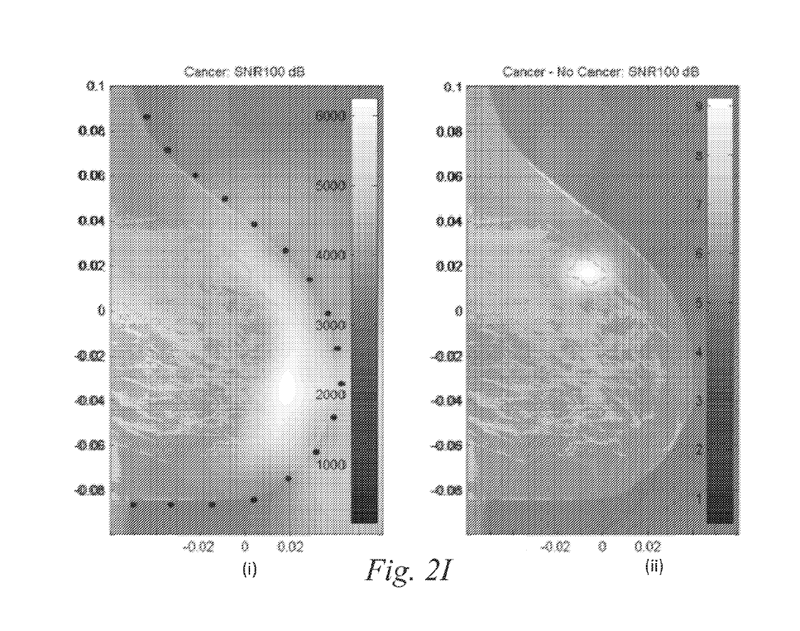

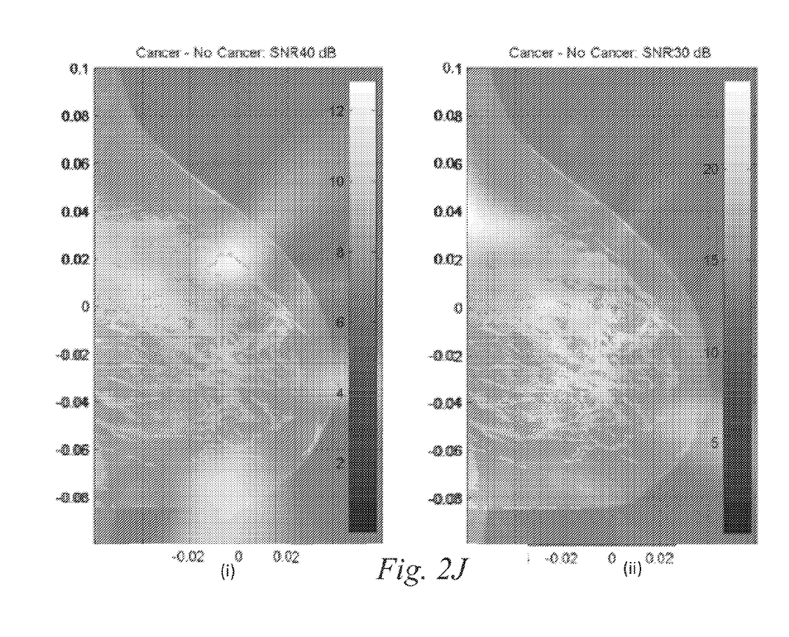

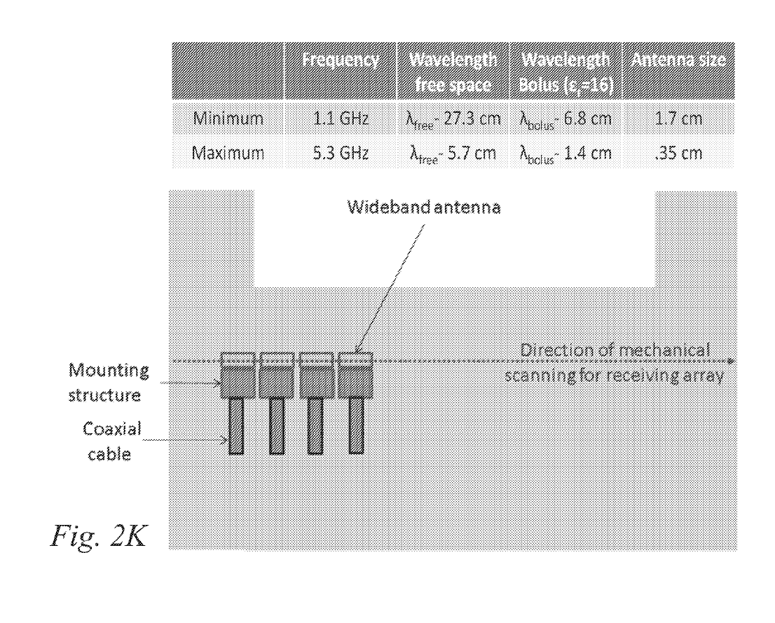

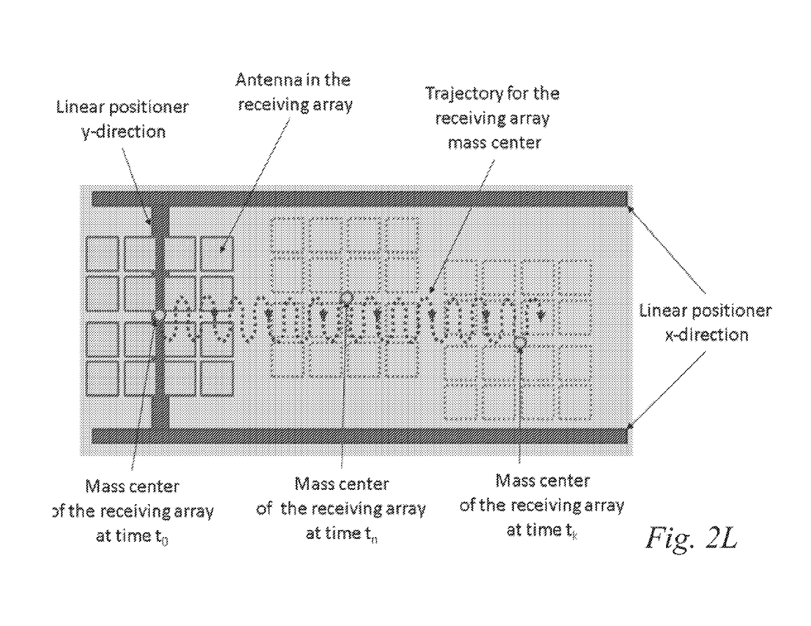

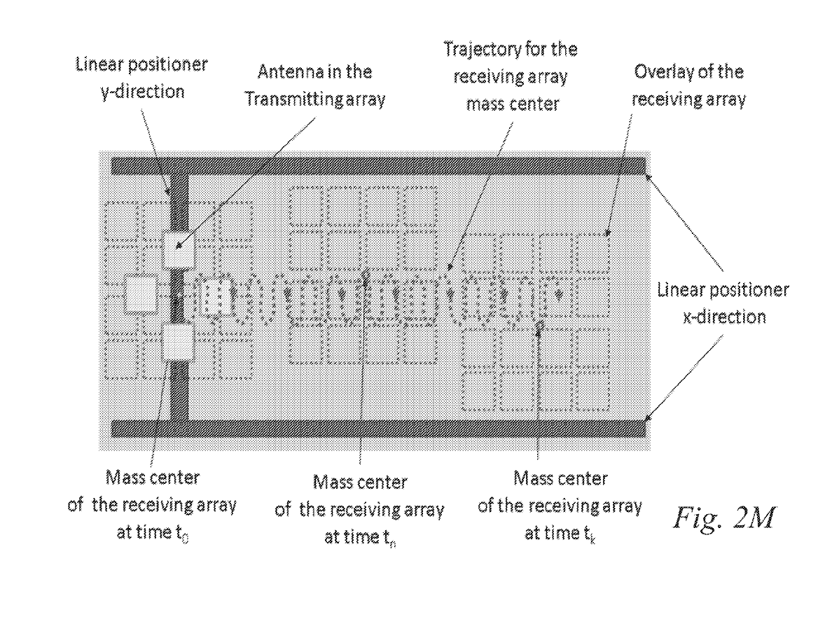

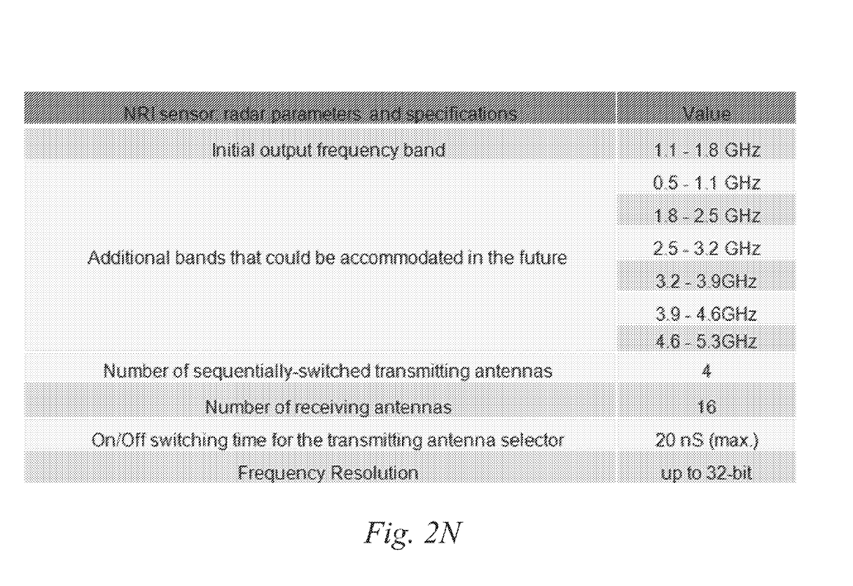

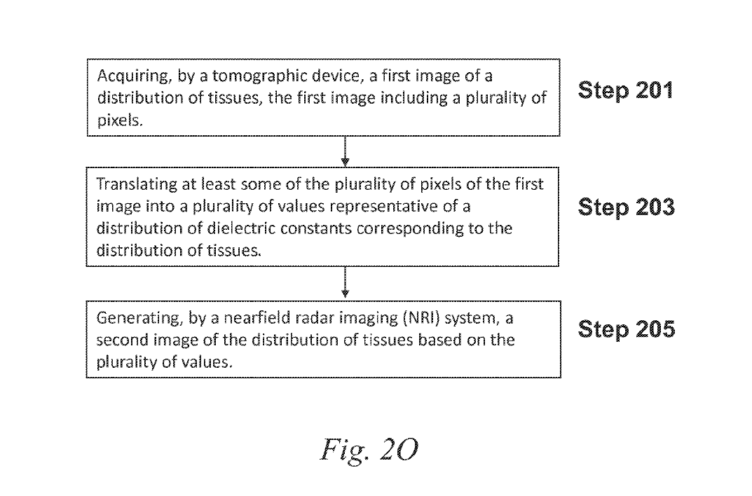

Referring to FIG. 2A, one embodiment of a system for granular imaging of a distribution of tissues is depicted. In brief overview, the system 211 may include one or more of subsystems or modules, for example, a tomographic device 230, an NRI system 290 and a tomographic image processor 280. Each of these subsystems or modules may be controlled by, or incorporate a computing device, for example as described above in connection with FIGS. 1A-1C. The system may sometimes be referred to as an imaging and processing system. The system may include or incorporate a compression or support structure 220 configured to support, hold, compress, contain and/or transport a distribution of tissues for imaging. The system 211 may be configured for detecting small-contrast anomalies, such as cancer tissue embedded in a diffuse matrix of fibroglandular healthy tissues, when a priori information about heterogeneous tissue distribution in a region (e.g., the breast) is provided by a tomographic method.

In some embodiments, the system 211 incorporates NRI imaging which provides significant contrast between fibroglandular and cancer tissue (e.g., in a distribution of breast tissues)—of the order of 10% for example—for microwave radar frequencies. Because certain distribution of tissues (e.g., in the breast) is may be highly heterogeneous with fibroglandular tissue randomly interspersed in the adipose background, solely using microwave imaging to detect breast cancer may provide limited success. In some body parts, the microwave contrast between fibroglandular and fatty tissue is even greater than that between fibroglandular and tumor tissue. The microwave scattering may become quite disorganized, and may result in poor inversion and imaging. The system can incorporate NRI and tomographic (e.g., DBT) methods, which may interoperate in a configuration such that the probability of detecting breast cancer is increased by (1) using a higher tumor/healthy tissue contrast available to NRI to supplement tomographic imaging and (2) incorporating a-priori information about the geometric configuration or distribution of fibroglandular tissue provided by the tomographic method in the NRI imaging algorithm.

In certain embodiments, by using a high resolution X-ray-based tomographic (e.g., DBT) images for example, the spatial organization of the breast tissues can be obtained and coupled simultaneously, or within a short predetermined time period, with observed microwave measurements to perform a three dimensional reconstruction. By computationally modeling the propagation and scattering of the incident field throughout the tomographic or DBT-imaged fibroglandular matrix, some embodiments of system 211 can help determine whether certain regions of high dielectric contrast represent normal fibroglandular or cancer tissue. Thus, tomographic and NRI technologies can be complementary and combined to improve breast cancer detection when operating together in a hybrid co-registered system. In order to incorporate the two technologies, mechanical and electrical adaptations, modifications, additions, accommodations and/or customizations are applied to link or incorporate the tomographic component to the NRI component.

Referring again to FIG. 2A, and in some embodiments, the system 211 includes a tomographic device 230. The tomographic device may be built, designed and/or configured to performing imaging of an object or mass by sections or sectioning, through the use of a penetrating wave such as X-ray. For example, in some embodiments, the tomographic device include an X-ray module for producing X-ray for imaging (e.g., X-ray computed tomography). However, some embodiments of the tomographic device may employ methods such as gamma rays (e.g., single-photon emission computed tomography), radio-frequency waves (e.g., magnetic resonance imaging), electron-positron annihilation (e.g., positron emission tomography), electrons (e.g., electron tomography or 3-dimensional transmission electron microscopy), muons (e.g., muon tomography), ions (e.g., atom probe) and magnetic particles (e.g., magnetic particle imaging). An image produced by the tomographic device may be referred to as a tomogram or tomographic image. In some embodiments, the tomographic device is configured to perform tomosynthesis and may be referred to as a tomosynthesis device or a digital breast tomosynthesis (DBT) device.

The tomographic device may perform laminographic imaging, e.g., using geometric or linear tomography. The tomographic device may acquire and/or generate two-dimensional (2D) image slices or data slices of an object at a high resolution (e.g., 85-160 micron), generally higher than computed tomography (CT). The tomographic device may generate, construct or provide a 3-dimensional (3D) image or representation (sometimes generally referred to as a tomographic image), e.g., from the image or data slices.

The tomographic device 230 may be part of an integrated single-pass, co-registered data collection setup (e.g., including NRI and tomographic sensors). The radiological contrast between cancer and fibroglandular tissue provided by the tomographic device may be supplemented by the NRI or microwave sensing, which can have an order of magnitude greater contrast (e.g., 10%) between cancer and fibroglandular tissue. In some embodiments, multiple tomographic or DBT projections can be acquired with the object. The object may comprise a distribution of tissues, which can include any biological, artificial, organic or inorganic tissues, fibers and/or materials (hereafter sometimes generally referred to as “distribution of tissues”). The object may include one or more material of any dielectric constant. The object may comprise any solid, semi-solid, viscous, liquid, suspension, vapor or gaseous element or a combination of elements.

By way of illustration, the object may include human tissue, such as a breast under clinical compression, e.g., using dielectrically-appropriate X-ray-transparent compression paddles. FIG. 2B depicts one embodiment of a tomographic device used to produce a 3D reconstruction (e.g., of a breast in a conventional DBT manner). FIG. 2C depicts one embodiment of an NRI system. NRI measurements may be collected quasi-simultaneously as the tomographic scan using microwave antennas located inside a plastic container filled with a bolus matching liquid. Such a configuration may enable or provide exact registration of the two imaging technologies. The NRI system can use a priori information provided by the tomographic sensor, comprising a 3D map or representation of the tissue distribution inside of the object (e.g., breast). This a priori information can enhance a first guess used during the imaging reconstruction.

In certain embodiments, the tomographic device incorporates or employs a DBT system and DBT methods. DBT is an X-ray imaging technology that can yield higher selectivity of breast cancer detection compared to conventional mammography. Instead of relying on the still limited (e.g., 1%) radiological contrast provided by DBT between cancer tissue and healthy, commonly-occurring fibroglandular tissue, certain embodiments of the system 211 may use the 3D DBT image to instead estimate or determine the object's tissue composition and/or distribution.

In some embodiments, the system 211 includes a tomographic image processor 280 (TIP) built, designed and/or configured for determining, estimating or inferring characteristics of the object's distribution of tissues. In certain embodiments, the microwave modeling software and/or NRI imaging algorithm of the NRI system, used for detecting cancer tissues may leverage on, rely on or require an accurate geometrical model of the tissue distribution inside of the object or breast. In some embodiments, the breast or object may be segmented into regions corresponding to one of the following types of tissues for example: 1) muscle tissue, 2) fibrous-connective and glandular tissue, 3) adipose tissue, and 4) skin. However, each pixel in the corresponding 3D model may not correspond to 100% muscle or 100% fibrous-connective and glandular or 100% adipose, or 100% skin. Rather, a pixel may comprise some mixture of these tissues, and accordingly, the segmentation method can lead to erroneous modeling and/or imaging results, since tissue composition of each pixel in the 3D model can vary significantly over a fine scale.

In some embodiments, and as an improvement, the TIP may use the tomographic image (e.g., 3D DBT image) generated by the tomographic device, to estimate or determine the object's tissue composition and/or distribution at a granular level. The tissue distribution may be analyzed, determined or examined down to the pixel level of the tomographic image. As described herein, the TIP can determine or infer the corresponding dielectric constants down to the pixel level in the 3D geometric model. The TIP can generate a representation of the dielectric constants, which can be used by the system's microwave modeling software and/or NRI imaging algorithm. For example, and in some embodiments, the amount of fat in each pixel of the 3D geometric model can be extracted from the attenuation coefficient of the tomographic (e.g., 3D DBT) image. FIG. 2D depicts example embodiments of representations of the distribution of tissues. For example, part (a) depicts one embodiment of a slice of a 3D Digital Breast Tomosynthesis image, and part (b) depicts one embodiment of the slice registered into four types of tissue: 1) muscle tissue; 2) fibrous-connective and glandular tissue; 3) adipose tissue; and 4) skin.

The TIP can combine information about the dielectric constants with predetermined experimental data (e.g., relating tissue types with dielectric constants) and/or with numerical models (e.g., relating tissue types with dielectric constants) to estimate or determine a dielectric constant corresponding to each of a plurality of pixels, as a function of the fat content for example. The TIP may translate or map fat content at a particular locality (e.g., pixel or pixels) to a dielectric constant. In other embodiments, water content or other type of characteristics may be used instead of fat content.

By way of example, it is possible for the TIP to determine as many dielectric constants as pixels that exist in the tomographic (e.g., 3D DBT) image, leading to an accurate and/or granular representation (or spatial distribution) of non-invasively measured dielectric constant values inside the object (e.g., breast). The TIP may extract or determine the amount of fat, for example, at each location or pixel of the 3D tomographic image or model. The TIP may translate the amount of fat to a specific value or representation of a corresponding dielectric constant. The TIP may generate or provide the dielectric constants for use in a microwave geometry model. There may be as many dielectric constant values as pixels in the 3D tomographic image, allowing for a refined model. The refined microwave geometry model can be used to produce accurate heterogeneous background information for the NRI algorithm process, so that performance of the breast cancer detection algorithm or process may be improved.

In certain embodiments, an average or effective dielectric constant in a region (e.g., in a pixel, or volume) of the 3D tomographic (e.g., DBT) image, may be inferred or determined based on fat content corresponding to the region. The TIP may determine dielectric constant values using one or more electromagnetic composite models. The TIP may use the fat-content F(rᵤ) of a pixel or region, located at rᵤ, in the tomographic image, to specify an electromagnetic composite model. In some embodiments, the electromagnetic composite model may comprise randomly distributing square pixels. f % of the pixels may be 100% fatty and (100-f) % may be 100% fibroconnective/glandular tissues, e.g., in a closed, square geometry. Overlaid on this geometry (e.g., as shown in FIG. 2E(i)) is the computed field distribution. By way of illustration, FIG. 2E(i) depicts one embodiment of an electric field (e.g., a total electric field) distribution superimposed on a composite model (E_{T}^{CM}), with fatty tissue dots in a square background of fibrous tissue, surrounded by air; FIG. 2E(ii) depicts one embodiment of an optimized homogeneous geometry or model (E_{T}^{H}(u)); and FIG. 2F depicts one embodiment of a difference between the electric field of the composite and the homogeneous models.

In some embodiments, the average complex dielectric constant of the composite model, specified by its dielectric permittivity Eₐᵥ^{CM} and its effective conductivity σₐᵥ^{CM}, may be unknown. The unknown values may be obtained by solving the following non-linear optimization problem, for example:

arg minᵤ∥E_{T}^{CM}−E_{T}^{H}(u)∥₂ (P1)

where u may be a vector containing the unknowns ∈ₐᵥ^{CM} and σₐᵥ^{CM}, E_{T}^{CM} may represent the (total) electric field—calculated as the addition of incident and scattered fields—produced by composite model, and E_{T}^{H}(u) may comprise the (total) field produced by a homogeneous geometry characterized by ∈ₐᵥ^{CM} and σₐᵥ^{CM}. The TIP may compute or determine the (total) fields of the composite model and homogeneous geometry using a full wave Finite Differences in the Frequency Domain (FDFD) method or any other full-wave electromagnetic model.

In some embodiments, and by way of non-limiting illustration, the composite model may be defined inside a 2 by 2 cm square, for example as shown in FIG. 2E(i). This model may be illuminated with a plane wave, working at 5 GHz for example, and propagating in the −y direction for example. The permittivity and conductivity of the 100% fatty tissue may be ∈_{100%}^{Fat}=3.19∈₀ [F/m] and σ_{100%}^{Fat}=0.113 [S/m], and the permittivity and conductivity of the 100% fibroconnective/glandular tissue is ∈_{100%}=57.3∈₀ [F/m] and σ_{100%}^{FCG}=0.114 [S/m], where ∈₀ is the permittivity of air. Each square pixel in the composite model, modeled as a 100% fat or 100% fibroconnective/glandular, is 0.05 by 0.05 cm. The total field produced by the composite model E_{T}^{CM} for a mixture of f=40% fat (60% fibrous tissue) is illustrated in one embodiment in FIG. 2E(i), and the total field produced by the optimized homogeneous geometry E_{T}^{H} (u) is illustrated in one embodiment in FIG. 2E(ii). The difference between the two total fields is depicted in one embodiment in FIG. 2F. The latter may show good agreement outside the composite/homogeneous region.

By way of illustration, the optimization procedure was performed for composite models having zero to nineteen percent of fat content, although other percentage ranges can be similarly performed. FIGS. 2G and 2H (in gray) depicts embodiments of plots of results obtained for the optimized average permittivity and dielectric constant respectively. These values are consistent with measured or known values (in black). The above process can produce an accurate map of the heterogeneous tissue distribution inside the breast, which can be used by an NRI algorithm to improve the effectiveness of breast cancer screening.

In some embodiments, the system 211 includes an NRI system 290 built, designed and/or configured to perform an NRI or radar-based scan of the object that is co-registered with the tomographic scan and/or tomographic image processing. The system 211 can perform accurate model-based imaging when an accurate spatial map of the dielectric constants of the breast or other object is used. The system 211 can perform model-based calculation of non-homogeneous Green's function inside the object. The NRI system may include a Finite Difference Frequency Domain (FDFD) electromagnetic code, or any other full-wave electromagnetic code or processing module (hereafter sometimes referred to as an “full wave modeling engine”). The full wave modeling engine may use the heterogeneous distribution of tissues to generate or compute a non-uniform, cancer-free Green's function associated with one or more antennas of the NRI system. The Green's function(s) may be used by the NRI system to produce cancer-free, background NRI synthetic data to be used by the NRI system's imaging algorithm.

In some embodiments, the NRI system includes an NRI imaging algorithm that is created, designed and/or configured to make accurate image reconstructions of the breast, e.g., in real time. The imaging algorithm may comprise a phase-based imaging algorithm. Certain embodiments of the imaging algorithm can be mathematically represented or summarized for example, as follows:

I(rᵤ)=Σ_{l,n,p}E(f^{l},rₜⁿ,rᵣ^{p})e^{j(Φ}^{₁}^{ⁿ}^{(f}^{^{l}}^{,r}^{ₜ}^{ⁿ}^{,r}^{ᵤ}^{)+Φ}^{₂}^{^{p}}^{(f}^{^{l}}^{,r}^{ᵣ}^{^{p}}^{,r}^{ᵤ}⁾⁾ (1)

where I(rᵤ) may represent an image at a point rᵤ inside the distribution of tissues. E(f^{l}, rₜⁿ, rᵣ^{p}) may comprise a vector, and may represent an electric field generated on at a p^{th} receiving antenna of the NRI system or sensor, located at rᵣ^{p}, when an n^{th} transmitting antenna of the NRI system or sensor, located at rₜⁿ, is radiating with a l^{th} frequency f^{l}. Φ₁ⁿ(f^{l}, rₜⁿ, r^{u}) may comprise a scalar and may represent a phase produced by the n^{th} transmitting antenna on an imaging point rᵤ. Φ₂^{p}(f^{l}, rᵣ^{p}, rᵤ) may comprise a scalar, and may be equivalent to Φ₁ⁿ, but for the p^{th} antenna. The phase terms, Φ₁ⁿ and Φ₂^{p}, may be obtained from a 3D full-wave electromagnetic solver (which may be part of the NRI system), which may be configured to predict the propagation of electromagnetic waves through complex non-homogeneous tissues (e.g., inside of the breast).

In some embodiments, the NRI system or the NRI imaging algorithm may be configured to implement or build a 3D Finite Difference in the Frequency Domain (3D-FDFD) full wave electromagnetic forward model capable of characterizing the interaction of electromagnetic waves with heterogeneous, lossy and/or dispersive tissues within a predetermined amount of time (e.g., within a reasonable amount of time for subjecting a patient to the imaging process). The NRI imaging algorithm may use the 3D-FDFD method to predict or model the propagation of electromagnetic fields inside of the distribution of tissues. For example, and in some embodiments, the NRI imaging algorithm may use the 3D-FDFD method to compute the Φ₁ⁿ and Φ₂^{p} terms, which may represent phases associated with the non-homogeneous Green's functions inside a cancer-free distribution of tissues (e.g., breast tissues).

In some embodiments, the NRI system may use an NRI sensor comprising a plurality of antennas (e.g., seventeen antennas). FIG. 2I(i) depicts one embodiment of a configuration of antennas of an NRI sensor. The antenna are shown as black points in the figure. The NRI system may incorporate an array of antennas that is intimately coupled to the breast. The NRI sensor may operate with the tomographic device for simultaneous data collection. Although the antennas are shown to conform to the outer contours of the object, in certain embodiments, the antennas may be arranged in a different configuration (e.g., a linear or grid array). The antennas may operate at a plurality of (e.g., eleven, equally spaced) frequencies (e.g., from 1-2 GHz). FIG. 2I depicts an example embodiment of an image with the contour of the cancer tissue marked with an outline. Parts (i) and (ii), for example, depict the phased-based image, superimposed on a given DBT slice, that may be obtained when the non-cancerous background field E^{NC} is present, and when subtracted from the cancerous field Ec for a signal-to noise ratio (SNR) of 100 dB.

Frequency-dependent tissue dielectric constants may be extracted from the fat content in the 3D tomographic image. FIG. 2I(i) depicts one embodiment of a phase-based image obtained when the 3D-FDFD-synthetically-generated field, E(f^{l}, rₜⁿ, rᵣ^{p})=E^{C} for the cancer case is used in Equation (1). The cancer tissue may not be detectable in this image, but, fortunately, it may be seen when the 3D-FDFD-synthetically-generated, cancer-free background field, E(f^{l}, rₜⁿ, rᵣ^{p})=E^{NC}, is subtracted from E^{C} (see, e.g., FIG. 2I(ii)). The pixel intensity of cancer tissue in the background-removed image may be less than 1% that of the non-background-removed image. In some embodiments, this indicates that high sensitivity and low noise levels is required for detecting cancer tissue.

By way of illustration, the impact of signal-to noise ratio (SNR) on the reconstructed image may be shown in FIG. 2J. Parts (i) and (ii), for example, depict embodiments of the phased-based image, superimposed on a given DBT slice, that may be obtained when the non-cancerous background field E^{NC} is subtracted from cancerous field E^{C}, for a signal-to noise ratio (SNR) of 40 dB and 30 dB respectively. The cancerous tissue appears to be still distinguishable in the image for the 40 dB case, but artifacts appearing through the image can make positive identification challenging. The image in the 30 dB case appears to show little or no correlation between the position of the cancerous tissue and the regions of high intensity values.

In some embodiments, the NRI system includes an NRI sensor capable of performing a mechanical scan, using two-axis linear positioners, and an electrical scan. The NRI system may include two arrays of transmitting and receiving antennas. The NRI system may be configured to collect microwave data at a suitable or required spatial and temporal sampling rate (see, e.g., FIG. 2C). In some embodiments, operation of the tomographic-NRI hybrid system may include collecting tomographic data within a predefined period of time (e.g., in about 5, 10 or 20 seconds). The system 211 may direct the NRI sensor to collect the microwave data within a predetermined period of time (e.g., in less than 5 or 10 seconds), and/or to do so within a predefined period of time from the tomographic scan. This procedure may utilize or require an amount of time similar to or less than a conventional mammography screening test.

In certain embodiments, the NRI system is used to collect microwave data right after, or within a short time after the tomographic imaging is complete. The breast or other distribution of tissues is held or maintained under the same shape, support, orientation and/or pressure (e.g., clinical compression) in both procedures or stages. Tight or accurate registration between the two modalities may permit an exchange of information between the two modalities, and can enhance the probability of breast cancer detection by the integrated system. In some embodiments, the NRI sensor may reside in and/or be enclosed in a plastic or other type of container. In other embodiments, the NRI sensor may be external to the container. The NRI sensor may be mechanically tailored to fit, for example, into the compression paddles (or support structures) of currently deployed tomographic (e.g., DBT) systems. In certain embodiments, the NRI sensor may be external to and/or decoupled with the support or compression structures for the distribution of tissues. In some embodiments, the NRI sensor may be coupled to the object while the object is tomographically scanned. For example, the NRI sensor may be designed to have a minimal footprint and/or corrected for introducing distortion to the tomographic image. In certain embodiments, at least some parts of the tomographic and NRI scans may overlap in time, e.g., to reduce the overall data acquisition time. The latter may reduce the time during which a patient may be subject to stress and/or discomfort (e.g., clinical compression of the breast).

In some embodiments, the container may host two arrays of transmitting and receiving antennas. The elements in the arrays may include wideband antennas, for example, so that multi-frequency tomographic reconstruction can be performed with the microwave data. In certain embodiments, the antenna element incorporates a spiral antenna design. The antenna elements may include wideband monopoles and/or fractal antennas, as further non-limiting examples. The plastic container may be filled with a matching liquid. The liquid may allow better coupling of electromagnetic radiation into the breast or other object. The liquid may help to miniaturize the antennas of the NRI sensor. For example, a suitable bolus liquid of relative dielectric constant of 16 can reduce the size of an antenna in free-space by a factor of 4, and can improve the packing of elements in the NRI sensor (see, e.g., FIG. 2K).

In some aspects, the antennas may be assembled in a mounting structure, and may be configured as a two dimensional array, which can provide three dimensional focusing capability to the system. The mounting structure may be connected to a two-axis linear positioner, in order to perform the mechanical-scanning as shown in FIG. 2K, for example. The mechanical-scanning can enhance imaging resolution and can minimize noise by enabling application of Synthetic Aperture Radar (SAR) algorithms. The antennas in the receiving array may be in a four by four configuration as shown in FIG. 2L for example. In some embodiments, the antennas in the transmitting array may be in a crossed configuration (e.g., with four antennas) as shown in FIG. 2M.

In some embodiments, the electromechanical scanning described herein can allow collection of microwave data in a dense grid of points. The scattered data can be finely measured across the entire object or breast area, compensating for the limitation on the density of bulky wideband antennas that can be packed into the available space. Thus, the spatial sampling requirement may be satisfied for both the highest and the lowest frequencies used by the system. This electromechanical scanning may provide a compromise solution balancing the electrical complexity of the arrays, the mechanical movement of the arrays, and the amount of data collected in a short amount of time.

In certain embodiments, the array of transmitting and receiving antennas may be connected to a microwave radar system. In some embodiments, the system 211 is scalable in frequency. Additional frequency bands can be added to the NRI sensor by for example changing the output frequencies of phase-locked sources (which may initially be operating at 2.5 GHz and 2.55 GHz for example), that drive an up-converter and a down-converter. This procedure may include adjusting band-pass filters (BPF) to a required frequency band. Some system-level specifications for one example embodiment of the NRI system are summarized in the table in FIG. 2N.

In some embodiments, the NRI imaging sensor provides optimal performance when the field that propagates through the breast or other object is measured with a spatial sampling rate of half a tissue wavelength outside the object. This may be implemented, for example, by arranging an array of transmitting and receiving antennas wrapped around the breast and collecting data with the specified spatial sampling rate. The implementation may not allow the wideband antennas of the NRI sensor working at microwave frequencies to satisfy the sampling criteria at the highest and lowest frequency. Since the minimal size of an effective antenna is of the order of a quarter to a half a wavelength, which implies that one microwave antenna of half a wavelength at the lowest frequency may not satisfy the sampling density requirement at the highest frequency, a mechanical solution for the spatial sampling requirement as described herein may be suitable and/or preferred. The mechanical configuration may include a pair of transmitting and receiving antennas that are mechanically scanned in order to collect the data with required spatial sampling rate. The time to mechanically scan the antennas may be reduced where possible to limit any stress or discomfort to the patient during the procedure.

In certain embodiments, the NRI sensor is built, designed and/or configured to balance the electrical and mechanical scanning capabilities of the sensor. The NRI/tomographic screening test may be performed at least as fast as a conventional mammography test. The NRI/tomographic system may include precisely custom-configured, conventional hardware combined with mathematical processing to provide an efficient screening technology that for example, can be widely accepted by health insurance companies. In some embodiments, the system includes a customized computational model and reconstruction algorithm for the dual modality NRUDBT configuration. The computational model may include a full-wave, three-dimensional, dispersive microwave propagation and scattering simulator that is suited for inhomogeneous distribution of tissues. A customized signal processing algorithm may be used to handle the microwave inversion and tissue geometry reconstruction.

Referring now to FIG. 2O, one embodiment of a method for granular imaging of a distribution of tissues is depicted. A tomographic device may acquire a first image of a distribution of tissues, the first image including a plurality of pixels (201). A tomographic image processor may translate at least some of the plurality of pixels of the first image into a plurality of values representative of a distribution of dielectric constants corresponding to the distribution of tissues (203). A nearfield radar imaging (NRI) system may generate a second image of the distribution of tissues based on the plurality of values (205).

Referring now to (201), and in some embodiments, a tomographic device of a system 211 may acquire a first image of a distribution of tissues. The first image may include a plurality of pixels. As discussed above in connection with at least FIG. 2A, the tomographic device may acquire and/or generate an image, such as but not limited to a DBT or CT image of an object comprising a distribution of tissues. The tomographic device may acquire the first image of an object comprising a distribution of tissues, the distribution of tissues comprising biological, artificial, organic or inorganic tissues, fibers and/or materials. The object may include one or more material of any dielectric constant. The distribution of tissues may for example comprise breast tissues or some other portion of a human body. The image may comprise a 3D image or model and/or one or more slices of 2D images. The image may comprise a plurality of pixels from the one or more 2D slices, or spatially-located pixels from the 3D image or model. The tomographic device may generate the first image comprising a high resolution X-ray-based tomographic image. The tomographic device may be configured to perform tomosynthesis on the distribution of tissues.

In some embodiments, the tomographic device is configured to generate and/or provide a priori information to an NRI system of the system 211. The tomographic device may be incorporated into an integrated single-pass, co-registered data collection setup with the NRI system. In certain embodiments, a support or compression structure of the system 211 may maintain at least one of: a shape or a size of the distribution of tissues, across time periods during which the first image is acquired and/or during which the distribution of tissues is electromagnetically scanned by the NRI system. The support or compression structure may ensure that the distribution of tissues is spatially maintained or substantially maintained between the tomographic and NRI processing for accurate registration. For example, the support or compression structure may comprise dielectrically-appropriate X-ray-transparent compression paddles, to place the object such as a breast under clinical compression. The support or compression structure may hold or support the distribution of tissues for a predetermined period of time, e.g., 5, 10, 15 or 20 seconds. The length of the predetermined period of time may be selected in part to minimize discomfort or stress to a corresponding patient undergoing imaging, and/or in part to allow tomographic and NRI processing.

The tomographic device may provide a priori information comprising a 3D map, geometrical model or representation of the tissue distribution inside of the object. The tomographic device may estimate or determine the object's tissue composition and/or distribution by segments or at a more granular level.

Referring now to (203), and in some embodiments, A tomographic image processor may translate at least some of the plurality of pixels of the first image into a plurality of values representative of a distribution of dielectric constants corresponding to the distribution of tissues. A tomographic image processor (TIP) may use the tomographic image generated by the tomographic device to determine, estimate or infer characteristics of the object's distribution of tissues. In some embodiments, the TIP may use the image to segment the distribution of tissues into regions corresponding to predefined types of tissues, for example as discussed above in connection with at least FIG. 2A. In certain embodiments, the TIP may analyze, determine or examine the tissue distribution at a granular level, e.g., down to the pixel level of the tomographic image.

In some embodiments, the TIP can determine or infer dielectric constants corresponding to the distribution of tissues down to the pixel level in the 3D geometric model. The TIP may translate at least some of the plurality of pixels of the first image into a plurality of values representative of a distribution of dielectric constants corresponding to the distribution of tissues. The TIP may translate at least some of the plurality of pixels of the first image into a plurality and/or distribution of dielectric constants corresponding to the distribution of tissues. The TIP can generate a representation or 3D map of the dielectric constants, which can be used by the system's microwave modeling software and/or NRI imaging algorithm. For example, and in some embodiments, the amount of fat in each pixel of the 3D geometric model can be extracted from the attenuation coefficient of the tomographic (e.g., 3D DBT) image.

The TIP may determine fat content corresponding to a first pixel of the plurality of pixels. The TIP may generate a first value of the plurality of values based on the determined fat content corresponding to the first pixel. In some embodiments, a number of pixels may be grouped into one of a plurality of regions in the object. The TIP may determine fat content corresponding to a first region of a plurality of regions within the distribution of tissues. The TIP may generate a first value of the plurality of values based on the determined fat content corresponding to the first region. The TIP can for example combine this fat content information with predetermined experimental data and/or with numerical models to estimate or determine a dielectric constant corresponding to each of a plurality of regions or pixels, as a function of the fat content for example.

The TIP may translate or map fat content at a particular locality (e.g., pixel or pixels) to a dielectric constant. In other embodiments, water content or other type of characteristics may be used instead of fat content. The TIP may extract or determine the amount of fat, for example, at each location or pixel of the 3D tomographic image or model. The TIP may translate the amount of fat to a specific value or representation of a corresponding dielectric constant. The TIP may generate or provide the dielectric constants for use in a microwave geometry model. The TIP can produce an accurate map of the heterogeneous tissue distribution inside the breast, which can be used by an NRI algorithm to improve the effectiveness of breast cancer screening.

Referring now to (205), and in some embodiments, a nearfield radar imaging (NRI) system may generate a second image of the distribution of tissues based on the plurality of values. The NRI system may perform an NRI or radar-based scan of the object that is co-registered with the tomographic scan and/or tomographic image processing. The NRI system may generate a second image comprising an NRI image using the a priori information. The NRI system may generate the second image based on the representation of the distribution of dielectric constants. The NRI system may electromagnetically scan the distribution of tissues within a predetermined period of time from the acquisition of the first image, e.g., to ensure accurate registration between the image acquisitions. As discussed above, the support or compression structure may also ensure that the distribution of tissues is spatially maintained or substantially maintained between the tomographic and NRI processing for accurate registration

In some embodiments, the NRI system may apply a Finite Difference Frequency Domain (FDFD) electromagnetic code or full wave modeling engine to the representation of the distribution of dielectric constants. The NRI system may generate (e.g., via the full wave modeling engine) a non-uniform function associated with at least one antenna of the NRI system. The NRI system may generate the non-uniform function based on a FDFD method. The full wave modeling engine may, for example, use the heterogeneous distribution of tissues to generate or compute a non-uniform, cancer-free Green's function associated with one or more antennas of the NRI system. The Green's function(s) may be used by the NRI system to produce cancer-free, background NRI synthetic data to be used by the NRI system's imaging algorithm.

The NRI system may apply an NRI imaging algorithm using the background data to generate accurate image reconstructions of the breast or other object, e.g., in real time. The imaging algorithm may comprise a phase-based imaging algorithm. The NRI imaging algorithm may implement or build a 3D Finite Difference in the Frequency Domain (3D-FDFD) full wave electromagnetic forward model capable of characterizing the interaction of electromagnetic waves with heterogeneous, lossy and/or dispersive tissues. The NRI imaging algorithm may use the 3D-FDFD method to predict or model the propagation of electromagnetic fields inside of the distribution of tissues. In some embodiments, the NRI imaging algorithm may use the 3D-FDFD method to compute phases associated with the non-homogeneous Green's functions inside a cancer-free distribution of tissues.

In some embodiments, the NRI system may use an NRI sensor comprising a plurality of antennas. In some embodiments, the NRI system may incorporate an array of antennas that is intimately coupled to the object. The NRI sensor may operate with the tomographic device for simultaneous data collection. In certain embodiments, the antennas may be arranged in a different configuration (e.g., a linear or grid array). The antennas may operate at a plurality of (e.g., equally spaced) frequencies (e.g., from 1-2 GHz). Frequency-dependent tissue dielectric constants may be extracted from the fat content in the 3D tomographic image. In some embodiments, the NRI system may subtract the 3D-FDFD-synthetically-generated, cancer-free background field from one corresponding to the cancer case.

In some embodiments, the NRI system includes an NRI sensor for performing a mechanical scan, using two-axis linear positioners, and an electrical scan. The NRI system may use two arrays of transmitting and receiving antennas. The NRI system may collect microwave data at a suitable or required spatial and temporal sampling rate. In some embodiments, operation of the tomographic-NRI hybrid system may include collecting tomographic data within a predefined period of time (e.g., in about 5, 10 or 20 seconds). The NRI sensor may collect the microwave data within a predetermined period of time (e.g., in less than 5 or 10 seconds), and/or do so within a predefined period of time from the tomographic scan.

In certain embodiments, the NRI system collects microwave data right after, or within a short time after the tomographic imaging is complete. By way of illustration, in the context of breast cancer screening, a breast or other distribution of tissues is held or maintained by a support or compression structure, under the same shape, support, orientation and/or pressure (e.g., clinical compression) in the tomographic and NRI procedures or stages. Tight or accurate registration between the two modalities can aid exchange of information between the two modalities. The NRI system may locate the distribution of tissues within a plastic or other type of container containing a bolus matching liquid. In some embodiments, the NRI sensor may be located within the container. In other embodiments, the NRI sensor may be located external to the container.

In certain embodiments, at least some parts of the tomographic and NRI scans may overlap in time, e.g., to reduce the overall data acquisition time, and/or to reduce the time during which a patient may be subject to stress and/or discomfort. In some embodiments, the NRI system performs electromechanical scanning as described above in connection with at least FIGS. 2A, 2K, 2L and 2M, to collect microwave data in a dense grid of points. A pair of transmitting and receiving antennas may be mechanically scanned in order to collect the data with required spatial sampling rate. In certain embodiments, the antennas in the receiving array of the NRI system may simultaneously measure the electromagnetic wave that propagates through the heterogeneous distribution of tissues. Data collected by each receiving antenna may be later used by an NRI reconstruction algorithm.

The NRI system may generate the second image of the distribution of tissues based on the plurality of values corresponding to the distribution of dielectric constants. The NRI system may generate an image with reduced distortion from features introduced by fat content in the distribution of tissues. The NRI system may generate an image with improved contrast between cancer tissue and healthy tissues. A user, detection algorithm or module may identify cancerous tissue or other feature/anomaly from the second image.

It should be understood that the systems described above may provide multiple ones of any or each of those components and these components may be provided on either a standalone machine or, in some embodiments, on multiple machines in a distributed system. In addition, the systems and methods described above may be provided as one or more computer-readable programs or executable instructions embodied on or in one or more articles of manufacture. The article of manufacture may be a floppy disk, a hard disk, a CD-ROM, a flash memory card, a PROM, a RAM, a ROM, or a magnetic tape. In general, the computer-readable programs may be implemented in any programming language, such as LISP, PERL, C, C++, C#, PROLOG, or in any byte code language such as JAVA. The software programs or executable instructions may be stored on or in one or more articles of manufacture as object code.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

We claim:

1. A method for granular imaging of a distribution of tissues, the method comprising:
(a) acquiring, by a tomographic device, a first image of a distribution of tissues, the first image including a plurality of pixels;
(b) translating at least some of the plurality of pixels of the first image into a plurality of values representative of a distribution of dielectric constants corresponding to the distribution of tissues; and
(c) generating, by a nearfield radar imaging (NRI) system, a second image of the distribution of tissues based on the plurality of values.

2. The method of claim 1, wherein (a) comprises acquiring the first image of the distribution of tissues, the distribution of tissues comprising breast tissues or some other portion of a human body.

3. The method of claim 1, further comprising identifying cancerous tissue or other feature from the second image.

4. The method of claim 1, further comprising electromagnetically scanning the distribution of tissues, by the NRI system, within a predetermined period of time from the acquisition of the first image.

5. The method of claim 1, further comprising maintaining at least one of: a shape or a size of the distribution of tissues, across time periods during which the first image is acquired and during which the distribution of tissues is electromagnetically scanned by the NRI system.

6. The method of claim 1, further comprising locating the distribution of tissues within a container containing a bolus matching liquid.

7. The method of claim 1, wherein (b) comprises determining fat content corresponding to a first pixel of the plurality of pixels.

8. The method of claim 7, further comprising generating a first value of the plurality of values based on the determined fat content corresponding to the first pixel.

9. The method of claim 1, wherein (c) comprises generating a non-uniform function associated with at least one antenna of the NRI system.

10. The method of claim 9, further comprising generating the non-uniform function based on a finite difference frequency domain (FDFD) method or other full-wave electromagnetic method.

11. A system for granular imaging of a distribution of tissues, the system comprising:
a tomographic device, the tomographic device configured to acquire a first image of a distribution of tissues, the first image including a plurality of pixels, wherein at least some of the plurality of pixels of the first image are translated into a plurality of values representative of a distribution of dielectric constants corresponding to the distribution of tissues; and
a nearfield radar imaging (NRI) system, the NRI system configured to generate a second image of the distribution of tissues based on the plurality of values.

12. The system of claim 11, wherein the tomographic device is configured to acquire the first image of the distribution of tissues, the distribution of tissues comprising breast tissues or some other portion of a human body.

13. The system of claim 11, wherein the second image is used to identify cancerous tissue or other feature.

14. The system of claim 11, wherein the NRI system is configured to electromagnetically scan the distribution of tissues within a predetermined period of time from the acquisition of the first image.

15. The system of claim 11, further comprising a compression or support structure configured to maintain at least one of: a shape or a size of the distribution of tissues, across time periods during which the first image is acquired and during which the distribution of tissues is electromagnetically scanned by the NRI system.

16. The system of claim 11, further comprising a container within which the distribution of tissues is located, the container containing a bolus matching liquid.

17. The system of claim 11, further comprising a tomographic image processor configured to determine fat content corresponding to a first pixel of the plurality of pixels.

18. The system of claim 17, wherein the tomographic image processor is configured to generate a first value of the plurality of values based on the determined fat content corresponding to the first pixel.

19. The system of claim 11, wherein the NRI system is configured to generate a non-uniform function associated with at least one antenna of the NRI system.

20. The system of claim 19, wherein the NRI system is configured to generate the non-uniform function based on a finite difference frequency domain (FDFD) method or other full-wave electromagnetic method.
